(19) United States
(12) Patent Application Publication
(10) Pub. No.: US 2024/0100310 A1
(43) Pub. Date: Mar. 28, 2024

(54) DEVICES, ASSEMBLIES, AND METHODS FOR DELIVERING AGENTS

(71) Applicant: Boston Scientific Scimed, Inc., Maple Grove, MN (US)

(72) Inventors: Andrew PIC, Northboro, MA (US); Christopher R. DEUEL, Melrose, MA (US); Aaron ROSENTHAL, Foxboro, MA (US); Ryan EVERS, Billerica, MA (US); Collin MURRAY, Bolton, MA (US); John B. GOLDEN, Norton, MA (US); Pauline Rosemary LIMBERG, Northborough, MA (US); Adam Gregory McDERMOTT, Lincoln, MA (US)

(73) Assignee: Boston Scientific Scimed, Inc., Maple Grove, MN (US)

(21) Appl. No.: 18/475,343

(22) Filed: Sep. 27, 2023

Related U.S. Application Data
(60) Provisional application No. 63/377,456, filed on Sep. 28, 2022.

(51) Int. Cl. A61M 31/00; A61M 39/22

(52) U.S. Cl. CPC A61M 31/00; A61M 39/22; A61M 2039/226; A61M 2202/0468

(57) ABSTRACT

A valve assembly for a medical device including a container including a first region and a second region, a first fluid inlet fluidly coupled to the first region, a second fluid inlet fluidly coupled to the second region, and a piston assembly disposed within the container. The container is configured to house an agent within the second region, the first fluid inlet is configured to deliver a first portion of pressurized fluid to the first region at a first pressure level, and the second fluid inlet is configured to deliver a second portion of pressurized fluid to the second region at a second pressure level that is less than the first pressure level. The piston assembly includes a valve configured to move from a first position to a second position in response to the piston assembly moving relative to the container.

### CROSS-REFERENCE TO RELATED APPLICATIONS

This application claims the benefit of priority of U.S. Provisional Patent Application No. 63/377,456, filed on Sep. 28, 2022, the entirety of which is incorporated herein by reference.

### TECHNICAL FIELD

Various aspects of this disclosure relate generally to devices and methods for delivering agents. More specifically, in embodiments, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a device inserted into the working channel of the endoscope. Agent delivery may be achieved, for example, through mechanical systems designed to allow the agent to flow continuously from the device to the remote site. Such systems, however, may require numerous steps or actuations to achieve delivery, or may not achieve a desired rate of agent delivery or a desired dosage of agent. Accordingly, these systems may be inefficient, may not achieve a desired rate of agent delivery, may result in the agent clogging portions of the delivery device, and/or may result in inconsistent dosing of the agent. The current disclosure may solve one or more of these issues or other issues in the art.

### SUMMARY

Each of the aspects disclosed herein may include one or more of the features described in connection with any of the other disclosed aspects. Aspects of the disclosure relate to, among other things, systems, devices, and methods for delivering an agent to a target treatment site using a medical device including a valve assembly. According to an example, a valve assembly for a medical device may include a container including a first region and a second region, where the container is configured to house an agent within the second region, a first fluid inlet fluidly coupled to the first region, the first fluid inlet configured to deliver a first portion of a pressurized fluid to the first region, the first portion of the pressurized fluid having a first pressure level, a second fluid inlet fluidly coupled to the second region, the second fluid inlet configured to deliver a second portion of the pressurized fluid to the second region, where the second portion of the pressurized fluid has a second pressure level that is less than the first pressure level, a piston assembly disposed within the container, the piston assembly configured to move relative to the container in response to the first region receiving the first portion of the pressurized fluid and the second region receiving the second portion of the pressurized fluid, where the piston assembly includes a valve that is configured to move from a first position to a second position in response to the piston assembly moving relative to the container, wherein, in the first position, the valve is configured to inhibit the agent from exiting the second region of the container, and in the second position, the valve is configured to guide the agent out of the second region of the container for delivery from the medical device.

Any of the valve assemblies described herein may include any of the following features. A first funnel positioned within the second region, wherein the first funnel is configured to receive the agent when the valve is in the first position. A second funnel positioned within the second region relatively below the first funnel, wherein the second funnel is configured to receive the agent from the first funnel when the valve is in the second position. The second funnel is in fluid communication with a source of the pressurized fluid via the second fluid inlet, and the second funnel includes a porous portion along a wall of the second funnel. The second funnel is configured to mix the agent received from the first funnel with the second portion of pressurized fluid received from the second fluid inlet through the porous portion. The second funnel is configured to guide a mixture of the agent and the second portion of pressurized fluid to a delivery conduit of the medical device that is in fluid communication with the second region of the container. The container is configured to generate the first pressure level of the first portion of pressurized fluid within the first region, and the second pressure level of the second portion of pressurized fluid within the second region, thereby forming a pressure differential between the first region and the second region. The piston assembly includes a piston that is at least partially disposed within the first region and a piston rod that is at least partially disposed within the second region, wherein the piston rod has a first end coupled to the valve and a second end coupled to the piston, and where the piston assembly includes a biasing mechanism configured to bias the piston towards a first direction, thereby moving the piston rod and the valve to the first position. In response to forming the pressure differential between the first region and the second region, the valve assembly is configured to counteract the bias generated against the piston by the biasing mechanism to move the piston rod and the valve from the first position to the second position. A tubing coupled to the second fluid inlet, the tubing is configured to deliver the second portion of pressurized fluid to the second fluid inlet, wherein the tubing includes at least one orifice that is configured to restrict a flow of the second portion of pressurized fluid through the second fluid inlet. The orifice is configured to regulate the second portion of pressurized fluid to the second pressure level, thereby generating the pressure differential between the first region and the second region. The orifice is configured to vent at least the second portion of pressurized fluid into a surrounding atmosphere of the medical device. The valve includes a plurality of channels configured to regulate a flow rate of the agent moving through the second region when the valve is in the second position. Each of the plurality of channels is separated from an adjacent channel by at least one of a plurality of ribs. Each of the plurality of ribs is configured to inhibit lateral movement of the piston rod relative to the second region of the container.

According to another example, a valve assembly for delivering an agent may include an actuator, a container configured to store an agent, a fluid inlet fluidly coupled to the container, a piston assembly disposed within and movable relative to the container, the piston assembly including a valve that is configured to release the agent from the container upon moving from a first position to a second position, and an actuation assembly coupled to the actuator, the piston assembly, and a source of pressurized fluid, wherein, in response to actuating the actuator, the actuation assembly is configured to: interact with the source of pressurized fluid, thereby releasing the pressurized fluid from the source into the container via the fluid inlet, and move the piston assembly within the container, thereby repositioning the valve from the first position to the second position and releasing the agent from the container, wherein the pressurized fluid is configured to mix with the agent within the container and guide a mixture of the agent and the pressurized fluid towards an outlet that is in fluid communication with the container.

Any of the valve assemblies disclosed herein may include any of the following features. The actuation assembly includes a cam coupled to the source of pressurized fluid and at least one movable rod coupled to the piston assembly, wherein the cam is configured to engage a seal of the source of pressurized fluid to release the pressurized fluid, and the at least one movable rod is configured to push the valve towards the second position. The valve includes a plurality of channels and a plurality of ribs disposed about a perimeter of the valve, wherein at least one of the plurality of ribs is positioned between an adjacent pair of the plurality of channels. The plurality of channels is configured to control a rate of flow of the agent moving through the valve when in the second position.

According to another example, a method for delivering an agent from a medical device may include releasing a pressurized fluid into a first region of a valve assembly of the medical device to pressurize the first region to a first pressure level, and a second region of the valve assembly to pressurize the second region to a second pressure level that is less than the first pressure level, wherein the second region houses the agent, generating a pressure differential between the first region and the second region in response to the first region receiving the pressurized fluid at the first pressure level and the second region receiving the pressurized fluid at the second pressure level, causing a piston assembly to move relative to the first region and the second region in response to the valve assembly generating the pressure differential between the first region and the second region, wherein the piston assembly includes a valve that is configured to move from a first position to a second position when the pressure differential is generated within the valve assembly, and releasing the agent from the second region when the valve is moved to the second position, thereby allowing the pressurized fluid and the agent to mix prior to delivery from the medical device.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The terms “top” and “upper” refer to a direction or side of a device relative to its orientation during use, and the term “bottom” and “lower” refer to a direction or side of a device relative to its orientation during use that is opposite of the “top” and “bottom.” The term “exemplary” is used in the sense of “example,” rather than “ideal.” The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device according to some embodiments.

FIG. 2 shows a cross-sectional view of an exemplary valve assembly of the delivery device of FIG. 1 according to some embodiments.

FIG. 3A shows a top view of a valve of the valve assembly of FIG. 2 according to some embodiments.

FIG. 3B shows a cross-sectional view of the valve of FIG. 3A, taken along the line 3B-3B of FIG. 3A, according to some embodiments.

FIG. 4A shows a first actuation position of the valve assembly of FIG. 2 according to some embodiments.

FIG. 4B shows a second actuation position of the valve assembly of FIG. 2 according to some embodiments.

FIG. 5A shows an exemplary actuation assembly of the delivery device of FIG. 1 according to some embodiments.

FIG. 5B shows a partial side view of a portion of the actuation assembly of FIG. 5A according to some embodiments.

### DETAILED DESCRIPTION

Embodiments of this disclosure relate to dispensing (delivery) devices having valve assemblies for selectively releasing an agent (e.g., a powdered agent) to a site of a medical procedure. The valve assembly may include a piston assembly and a valve that may collectively be configured to release an agent stored in the delivery device upon moving between a first position and a second position in response to the valve assembly receiving a pressurized fluid (e.g., a gas) from a pressurized medium source (e.g., a gas canister). The agent may be received within an enclosure of the dispensing device, and in fluid communication with the pressurized fluid through one or more inlets of the valve assembly.

Accordingly, when the valve is selectively moved by the piston assembly from the first position to the second position, which is in fluid communication with the pressurized fluid source, the agent may be released from the enclosure and exposed to the pressurized fluid. The agent may be agitated upon interacting with the pressurized fluid prior to delivery to a target site of the medical procedure. Aspects of the dispensing device and valve assembly, such as the piston assembly and the valve, may facilitate a controlled fluidization (e.g., agitation) of the agent with the flow of pressurized fluid prior to the agent being delivered, which may assist in selectively controlling the flow of agent out of the dispensing device to help to prevent or minimize clogging during delivery.

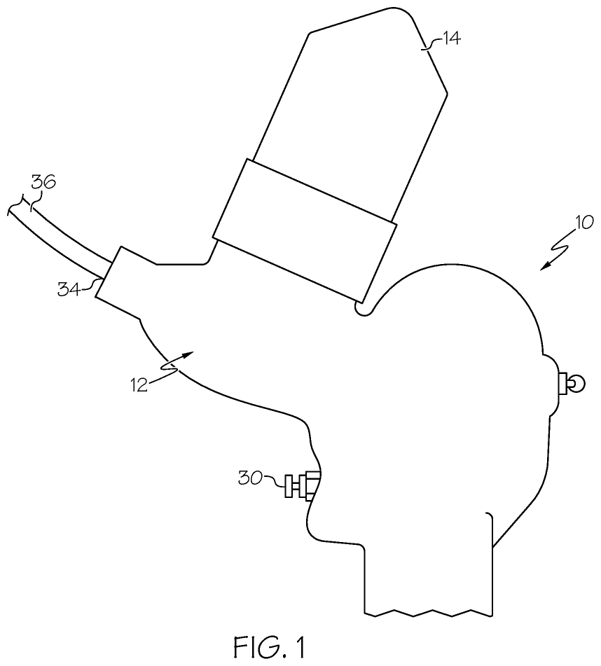

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. patent application Ser. No. 16/589,633, filed Oct. 1, 2019, published as U.S. Patent Application Publication No. 2020/0100986 A1 on Apr. 2, 2022, the disclosure of which is hereby incorporated by reference in its entirety, discloses features of exemplary delivery devices and systems. The features of this disclosure may be combined with any of the features described in the above-referenced application. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source in fluid communication with delivery system 10. Actuation mechanism 30 may be selectively actuated (e.g., manually depressible) or otherwise moved or actuated to control delivery of a material (e.g., a powdered agent) and pressurized fluid. The pressurized fluid alone, or a combination of a powdered agent and fluid, may be delivered from an outlet 34 of handle body 12. Outlet 34 may be in fluid communication with a delivery conduit, for example catheter 36 or another component for delivering the combination of agent and fluid to a desired location within a body lumen of a patient.

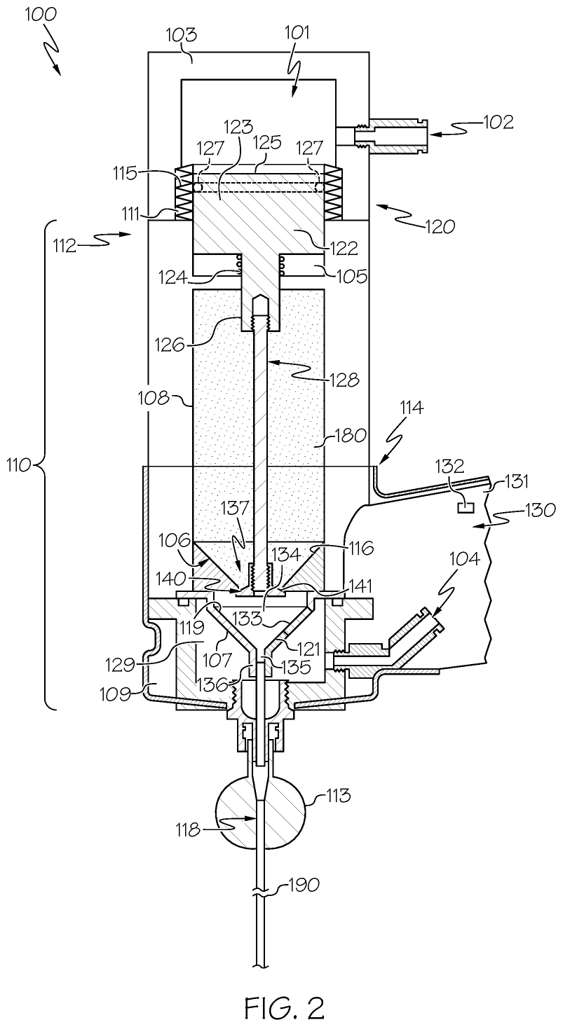

FIG. 2 shows aspects of an exemplary valve assembly 100. Valve assembly 100 may be housed within handle body 12 of delivery system 10, and particularly within enclosure 14. In some embodiments, valve assembly may be coupled to handle body 12 in lieu of enclosure 14. Valve assembly 100 may be selectively actuated when a pressure differential is generated within valve assembly 100. The pressure differential may be generated by an introduction of a pressurized fluid into valve assembly 100, which may be selectively introduced into valve assembly 100 via actuation mechanism 30 (see FIG. 1). Although not shown, actuation mechanism 30 may include one or more other actuation elements, such as, for example, a trigger, a button, a slider, a lever, a knob, a dial, and various other suitable actuators. As described herein, actuation of actuation mechanism 30 may provide for a corresponding release of pressurized fluid towards, and/or movement of, valve assembly 100, thereby controlling a delivery of agent through valve assembly 100 and toward a target treatment site in a patient.

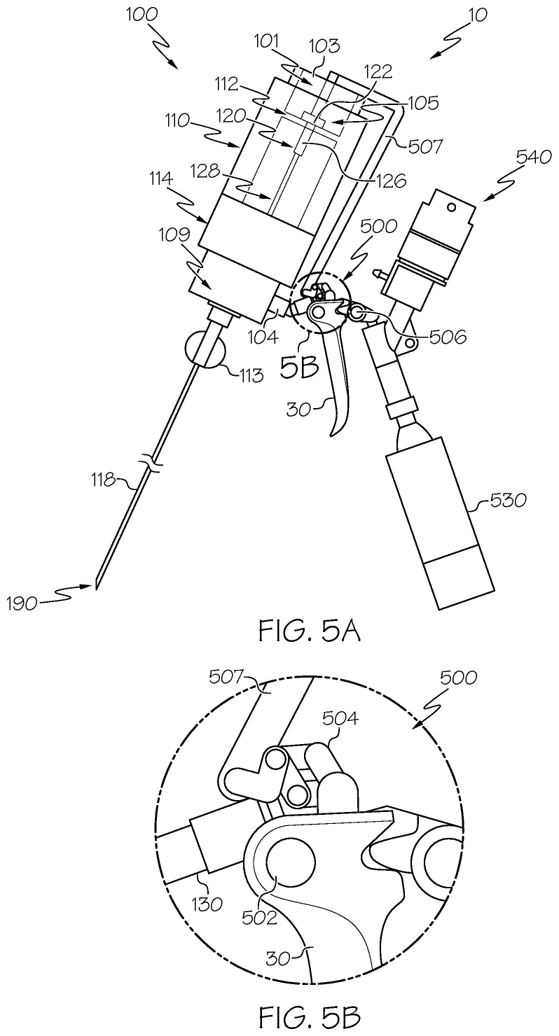

Alternatively, as described in detail below, actuation mechanism 30 may be coupled to valve assembly 100 by a mechanical actuation assembly that is configured to selectively actuate valve assembly 100 (see FIGS. 5A-5B). As described herein, the mechanical actuation assembly may include one or more cables, wires, rods, and/or various other suitable mechanisms for releasing pressurized fluid towards, and/or moving one or more internal components of, valve assembly 100.

Still referring to FIG. 2, valve assembly 100 may include a first (top) container 103 and a second (bottom) container 110 coupled to one another. First container 103 may define a first region of valve assembly 100, and second container 110 may define a second region of valve assembly 100. First container 103 and second container 110 may collectively form a canister housing defining an enclosure for storing an agent 180. Therefore, although described herein as separate containers, it should be understood that first container 103 and second container 110 may define subcomponents of a single canister housing.

Second container 110 may include a bottom portion 109, a top portion 112 that is opposite of bottom portion 109, and an intermediate portion 114 disposed between bottom portion 109 and top portion 112. In the example, at least one or more of intermediate portion 114 and/or bottom portion 109 of second container 110 may define the second region of valve assembly 100. First container 103 may include a cavity 101, and second container 110 may include a first (upper) cavity 105 along top portion 112, a second (intermediate) cavity 108 along intermediate portion 114, and a third (lower) cavity 134 within bottom portion 109. It should be appreciated that first cavity 105, second cavity 108, and third cavity 134 of second container 110 may be in fluid communication with one another. As described herein, cavity 101 of first container 103 may be fluidly isolated from the cavities of second container 110, such as by a gasket positioned between cavity 101 and first cavity 105.

In some embodiments, at least a portion of cavity 101 may define a high pressure zone of valve assembly 100, and one or more of first cavity 105, second cavity 108, and/or third cavity 134 may define a low pressure zone of valve assembly 100. In one example, first cavity 105 may define a portion of the low pressure zone. Alternatively, in other embodiments, cavity 101 and at least a portion of first cavity 105 may collectively define the high pressure zone of valve assembly 100.

Still referring to FIG. 2, second container 110 may be configured to store an agent within second cavity 108, such as agent 180 (e.g. a powder). In some embodiments, each of first container 103 and second container 110 may include complementary connection interfaces enabling a bottom portion of first container 103 to be selectively coupled onto top portion 112 of second container 110. For example, an outer surface of second container 110 may include a threaded portion 111 along top portion 112, and an inner surface of first container 103 may include a corresponding threaded portion 115 for engaging threaded portion 111 of second container 110. However, various suitable connection mechanisms and/or interfaces for joining first container 103 to second container 110 may be utilized without departing from a scope of this disclosure.

First container 103 may be fluidly coupled to a first fluid inlet 102 that is in fluid communication with a pressurized medium source (not shown), such that valve assembly 100 may be configured to receive a pressurized fluid at first container 103 via first fluid inlet 102. As described herein, first fluid inlet 102 may define a high pressure fluid inlet of valve assembly 100. Second container 110 may be fluidly coupled to a second fluid inlet 104 that is in fluid communication with the pressurized medium source, such that valve assembly 100 may be configured to receive the pressurized fluid at second container 110, and particularly a chamber 129 of bottom portion 109, via second fluid inlet 104. As described herein, second fluid inlet 104 may define a low pressure fluid inlet of valve assembly 100.

In some embodiments, valve assembly 100 may be configured to simultaneously receive the pressurized fluid at cavity 101 of first container 103 and third cavity 134 of second container 110. In other embodiments, valve assembly 100 may be configured to selectively receive the pressurized fluid through each of first container 103 and second container 110. In some embodiments, with bottom portion 109 in fluid communication with intermediate portion 114, second cavity 108 may be configured to receive the pressurized fluid from second fluid inlet 104.

Although fluid inlets 102, 104 are shown along a respective sidewall of first container 103 and second container 110, respectively, it will be appreciated that fluid inlets 102, 104 may be positioned at various alternative locations relative to valve assembly 100 without departing from a scope of this disclosure. In some embodiments, an inner and/or outer wall of first fluid inlet 102 may include an interface (e.g., a lip, a protrusion, a tab, a recess, etc.) configured to facilitate a connection between first fluid inlet 102 and a tubing (not shown) that is in fluid communication with the pressurized medium source. Accordingly, the tubing may fluidly couple first container 103 with the pressurized medium source for delivering the pressurized fluid into cavity 101. An inner and/or outer wall of second fluid inlet 104 may include an interface (e.g., a lip, a protrusion, a tab, a recess, etc.) configured to facilitate a connection between second fluid inlet 104 and a tubing 130 that is in fluid communication with the pressurized medium source. Accordingly, tubing 130 may fluidly couple second container 110 with the pressurized medium source for delivering the pressurized fluid into third cavity 134.

Tubing 130 may include an opening and/or orifice 132 extending through a sidewall 131 of tubing 130. Orifice 132 may establish fluid communication between tubing 130, and specifically a lumen of tubing 130, and an area exterior of tubing 130 that is exposed to atmospheric pressure. As described herein, valve assembly 100 may be configured to generate a ratio and/or differential of high pressure fluid and low pressure fluid between first container 103 and second container 110 based on a pressure level of the fluid received at first fluid inlet 102 and second fluid inlet 104, respectively.

Specifically, orifice 132 may be configured to restrict and/or regulate a flow of the pressurized fluid entering second container 110 through second fluid inlet 104, while the flow of pressurized fluid entering first container 103 may remain relatively unrestricted and/or unregulated. Accordingly, due to the restriction of flow of the pressurized fluid received in second container 110 (and particularly chamber 129, third cavity 134, second cavity 108, and first cavity 105), valve assembly 100 may be configured to form the low pressure zone within one or more of first cavity 105, second cavity 108, third cavity 134, and/or chamber 129. Due to the unrestricted flow of pressurized fluid received in first container 103, valve assembly 100 may be configured to form the high pressure zone within cavity 101. In other words, valve assembly 100 may allow a greater pressure level of fluid to build within cavity 101 relative to at least cavity 134, thereby forming the high pressure zone and the low pressure zone, respectively.

It should be appreciated that a pressure level within the low pressure zone may vary relative to one or more of first cavity 105, second cavity 108, third cavity 134, and/or chamber 129. For example, first cavity 105 may have a lower relative pressure level than one or more of second cavity 108, third cavity 134, and/or chamber 129. By way of further example, third cavity 134 and/or chamber 129 may have a greater relative pressure level than one or more of first cavity 105 and/or second cavity 108.

Accordingly, orifice 132 may be configured to restrict the flow of pressurized fluid received in valve assembly 100 through second fluid inlet 104 such that a pressure level of the fluid received in tubing 130 may be controlled (e.g., decreased) by releasing a portion of the fluid through orifice 132, such as into a surrounding atmosphere of delivery system 10. With the tubing (not shown) coupled to first fluid inlet 102 not including an orifice, a pressure level of the pressurized fluid received within first container 103 (and specifically cavity 101) may be greater than the pressure level of the pressurized fluid received within second container 110 (and specifically first cavity 105, second cavity 108, third cavity 134, and/or chamber 129). The difference in pressure level may form the high pressure zone within valve assembly 100 at cavity 101, and the low pressure zone at first cavity 105, second cavity 108, third cavity 134, and/or chamber 129. As described herein, the high pressure zone and low pressure zone may be separated from another by a one or more devices of valve assembly 100 (e.g., a piston 122 and a gasket 127).

In some embodiments, a size and/or shape of orifice 132 may be determined based on a desired pressure differential generated within valve assembly 100 between the high pressure zone and the low pressure zone. For example, orifice 132 may be sized and/or shaped such that orifice 132 is configured to restrict a flow rate and/or pressure level of the pressurized fluid received in second container 110 to a range of about 800 liquid ohms to about 1200 liquid ohms, such as 1000 liquid ohms. A nominal pressure level within valve assembly 100 may range from about 5 psi to about 75 psi.

Still referring to FIG. 2, valve assembly 100 may include a first (upper) funnel 106 and a second (lower) funnel 107. For example, first funnel 106 may be disposed within second cavity 108, and second funnel 107 may be disposed within third cavity 134. First funnel 106 may be in fluid communication with second cavity 108, such that agent 180 may flow from second cavity 108 onto first funnel 106 (e.g., by gravitational forces). As described herein, first funnel 106 may include a center opening 137 that defines a valve seat for receiving one or more components of valve assembly 100 thereon (e.g., a valve 140). In some embodiments, second funnel 107 may be in fluid communication with a tubing 118, which may be fluidly coupled to a bottom end 136 of second funnel 107. Tubing 118 may include a connector 113 that is operable and configured to attach tubing 118 to valve assembly 100. Connector 113 may have a bulbous shape defining one or more flanges to facilitate grasping and/or manipulating tubing 118 during connection to valve assembly 100, and particularly bottom portion 109. It should be appreciated that connector 113 may have various other shapes and/or sizes that may be suitable to provide a surface for grasping and facilitating a connection of tubing 118 to valve assembly 100.

In some embodiments, first funnel 106 may include a wall 116 having a generally conical or funnel shape; however, in other embodiments, wall 116 may have various other suitable shapes and/or configurations without departing from a scope of this disclosure. Wall 116 may be sized and/or shaped to form a varying cross-sectional profile between a top end and a bottom end of first funnel 106. In other words, wall 116 may be tapered radially-inward towards center opening 137. Accordingly, as agent 180 moves downward through first funnel 106, agent 180 may encounter portions of first funnel 106 that vary in size, which may assist in reducing a clogging of agent 180 within second container 110. Agent 180 may be prone to bridging, which may result in clogging absent the variations in size of first funnel 106. Stated differently, first funnel 106 may be configured to inhibit a packing and/or clogging of agent 180 within second container 110 by guiding agent 180 along wall 116 and toward center opening 137.

Still referring to FIG. 2, second funnel 107 may include a wall 119 that has a generally conical or funnel shape. Wall 119 may be sized and/or shaped to form a varying cross-sectional profile between a top end and bottom end 136 of second funnel 107. In other words, wall 119 may be tapered radially-inward towards a center opening 135 of second funnel 107. As such, third cavity 134 may have a greater diameter at the top end of second funnel 107 relative to bottom end 136. An inner surface 133 of wall 119 may at least partially define third cavity 134 for receiving agent 180 from first funnel 106 (e.g., by gravitational forces), and third cavity 134 may terminate at center opening 135. As agent 180 moves downward through second funnel 107, agent 180 may encounter portions of third cavity 134 that vary in size, which may assist in reducing a clogging of agent 180 within third cavity 134.

As described above, agent 180 may be prone to bridging, which may result in clogging absent the variations in diameter of third cavity 134. As described herein, agent 180 may be at least partially received within third cavity 134 from second cavity 108 in response to a corresponding movement of one or more components of valve assembly 100 (e.g., valve 140). Although first funnel 106 and second funnel 107 are shown and described herein as having substantially similar shapes and/or cross-sectional profiles, it should be appreciated that each funnel may have a varying shape and/or size relative to one another without departing from a scope of this disclosure.

Still referring to FIG. 2, wall 119 may have a constant thickness between the top end and bottom end 136 of second funnel 107. In other embodiments, wall 119 may have a varying thickness. Additionally, tubing 118 may be coupled to second funnel 107 at bottom end 136, such that tubing 118 may be in fluid communication with third cavity 134 via center opening 135. Tubing 118 may be coupled to second funnel 107 via various suitable mechanisms, including, but not limited to, an adhesive, a frictional fit, one or more complementary interfaces (e.g., ridges, tabs, protrusions, grooves), and more. Tubing 118 may include an outlet 190 that is in fluid communication with catheter 36 (see FIG. 1). Accordingly, as described herein, agent 180 and the pressurized fluid received within second funnel 107 may be guided through tubing 118 (via center opening 135) and delivered to the patient via catheter 36.

In some embodiments, wall 119 may include a sintered and/or porous portion 121. For example, porous portion 121 may include a plurality of pores and/or passages formed between an outer surface of wall 119 and inner surface 133. Porous portion 121 may be configured to receive a pressurized fluid therethrough, while remaining portions of wall 119 may exclude pores and/or passages. In other words, the plurality of pores and/or passages may be positioned only along porous portion 121. In the example, porous portion 121 may be positioned relatively adjacent to (e.g., facing towards) second fluid inlet 104. In other embodiments, two or more portions of wall 119 may be sintered and/or porous. In further embodiments, a substantial portion of wall 119 may be sintered and/or porous, such that the plurality of pores and/or passages may be formed along an entirety of wall 119. Each of the plurality of passages may be sized, shaped, and otherwise configured to permit the pressurized fluid from second fluid inlet 104 to be received through wall 119, and inhibit agent 180 from exiting third cavity 134 via wall 119.

Still referring to FIG. 2, the upper end of second funnel 107 may be at least partially disposed in bottom portion 109 and positioned relatively underneath first funnel 106. Further, second funnel 107 may be fluidly coupled to intermediate portion 114 via first funnel 106. In the example, the upper end of second funnel 107 may be coupled to, and configured to form a seal with, first funnel 106 and/or one or more surfaces defining intermediate portion 114 of second container 110. In some examples, valve assembly 100 may include one or more sealing mechanisms (e.g., O-rings) positioned between an interface of second funnel 107 and first funnel 106 to fluidly couple second funnel 107 to second cavity 108, and particularly first funnel 106 disposed therein. With second funnel 107 coupled to second cavity 108, agent 180 may move from second cavity 108 to second funnel 107 via first funnel 106. As described herein, valve assembly 100 may be configured to deliver a mixture of agent 180 (stored in second cavity 108) and pressurized fluid (received at third cavity 134) to a patient through second funnel 107.

Still referring to FIG. 2, valve assembly 100 may include piston assembly 120. Piston assembly 120 may include a piston 122, a piston rod 128, and a valve 140. Piston 122 may be disposed within one or more of first container 103 and second container 110. Piston 122 may include an upper body 123 at least partially received in each of cavity 101 and first cavity 105, and a lower body 126 received in second cavity 108. Upper body 123 may have a cross-sectional dimension that is relatively greater than a cross-sectional dimension of lower body 126. In some embodiments, a first (top) end of piston rod 128 may be coupled to lower body 126 of piston 122 within second cavity 108, and a second (bottom) end of piston rod 128 may be coupled to a top end of valve 140 within second cavity 108. In some embodiments, piston 122 may include an internal threaded portion along lower body 126, and piston rod 128 may include an external threaded portion on the first (top) end for threadably coupling with the internal threaded portion of lower body 126. Piston rod 128 may further include an external threaded portion on the second (bottom) end for threadably coupling with an internal threaded portion of valve 140.

Valve 140 may include a top portion or end 141 that is sized, shaped, and/or otherwise configured to interface with first funnel 106, and particularly center opening 137 (e.g., the valve seat of first funnel 106). In the example, top end 141 may be angled relative to a longitudinal axis of piston rod 128. As described herein, valve 140 may be configured to move between a first (closed) position, in which valve 140 is engaged with at least a portion of first funnel 106 (e.g., the valve seat), and a second (open) position in which valve 140 is disengaged from first funnel 106 and extending into third cavity 134. Alternatively, valve 140 may be disengaged from first funnel 106 upon moving (e.g., retracting) upward into second cavity 108. Valve 140 may be configured to allow the agent 180 received on first funnel 106 to move toward second funnel 107 (e.g., by gravitational forces) upon moving from the first (closed) position to the second (open) position.

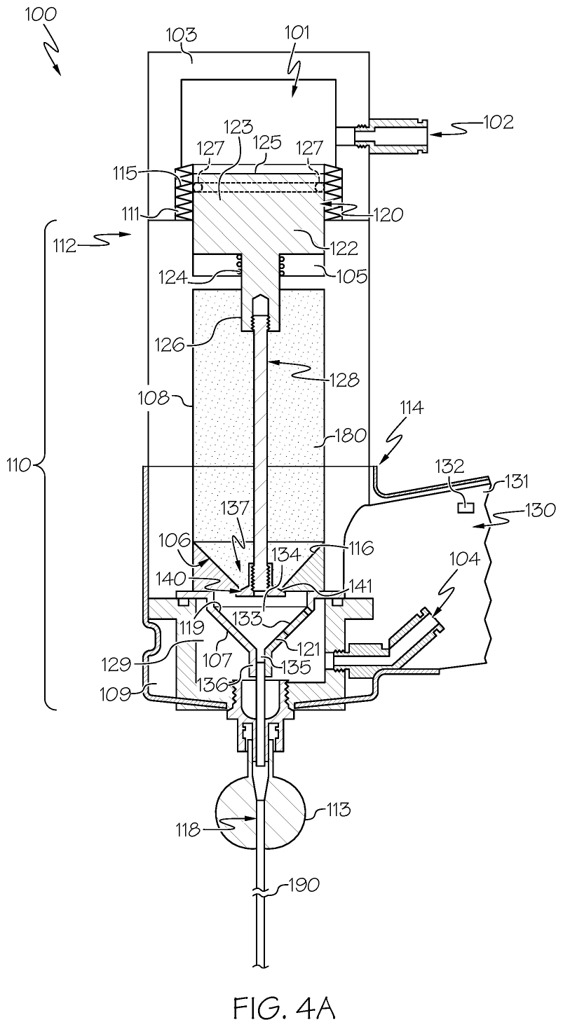

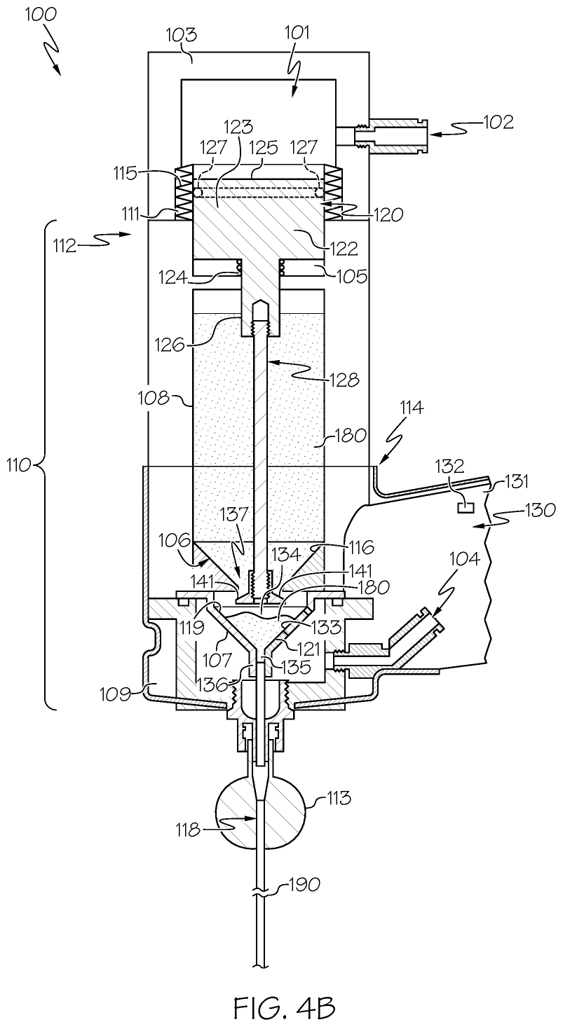

Piston assembly 120 may be configured to transition piston 122, piston rod 128, and valve 140 between one or more configurations in response to valve assembly 100 receiving a pressurized fluid from a pressurized medium source (see FIGS. 4A-4B). For example, piston assembly 120 may be actuated to move piston 122, piston rod 128, and valve 140 between one or more positions relative to first container 103, second container 110, and/or funnels 106, 107. As described herein, piston assembly 120 may be actuated in response to valve assembly 100 receiving a pressurized fluid from the pressurized medium source of delivery system 10 through each of first fluid inlet 102 and second fluid inlet 104.

Still referring to FIG. 2, piston assembly 120 may further include at least one biasing mechanism 124 disposed within first cavity 105 of top portion 112. Biasing mechanism 124 may be coupled to piston 122, and particularly a lower end of piston 122, such that biasing mechanism 124 may be disposed between the lower end of piston 122 and an interior (bottom) surface of top portion 112 that defines first cavity 105. Biasing mechanism 124 may be configured to urge piston 122 in a first (upward) direction relative to first cavity 105, thereby pushing piston 122 away from the interior (bottom) surface defining first cavity 105. Valve assembly 100 may include at least one gasket 127 coupled to piston 122 adjacent to a top surface 125 of piston 122. Gasket 127 may be disposed about an exterior of piston 122, and may be configured to inhibit fluid (e.g., gas) from moving between the opposing ends of piston 122. Accordingly, gasket 127 may inhibit fluid communication between cavity 101 and first cavity 105. Biasing mechanism 124 may be configured to bias piston 122, and thereby valve 140 coupled thereto via piston rod 128, to a first position (see FIG. 4A). As shown and described below, valve 140 may be configured to engage a portion of first funnel 106 (e.g., a valve seat at center opening 137) when piston 122 is in the first position.

Alternatively, in embodiments where valve 140 may disengage first funnel 106 by retracting upward into second cavity 108, biasing mechanism 124 may be positioned relatively above piston 122, such as within cavity 101. In this instance, biasing mechanism 124 may be configured to urge piston 122 in a downward direction relative to first cavity 105, thereby pushing piston 122 toward the interior (bottom) surface that defines first cavity 105.

Piston assembly 120 may be configured to compress biasing mechanism 124 in response to an actuation of actuation mechanism 30 and receipt of pressurized fluid in cavity 101 (via first fluid inlet 102) and third cavity 134 (via second fluid inlet 104). The pressurized fluid received in valve assembly 100 at first container 103 and second container 110 may generate a resulting pressure differential between first container 103 (e.g., the first region) and second container 110 (e.g., the second region). In this instance, the generated pressure differential may cause piston 122, piston rod 128, and valve 140 to move downward relative to first cavity 105, second cavity 108, and third cavity 134, such as to a second position (see FIG. 4B). As shown and described below, valve 140 may be configured to disengage a portion of first funnel 106 (e.g., the valve seat) when piston 122 is moved to the second position.

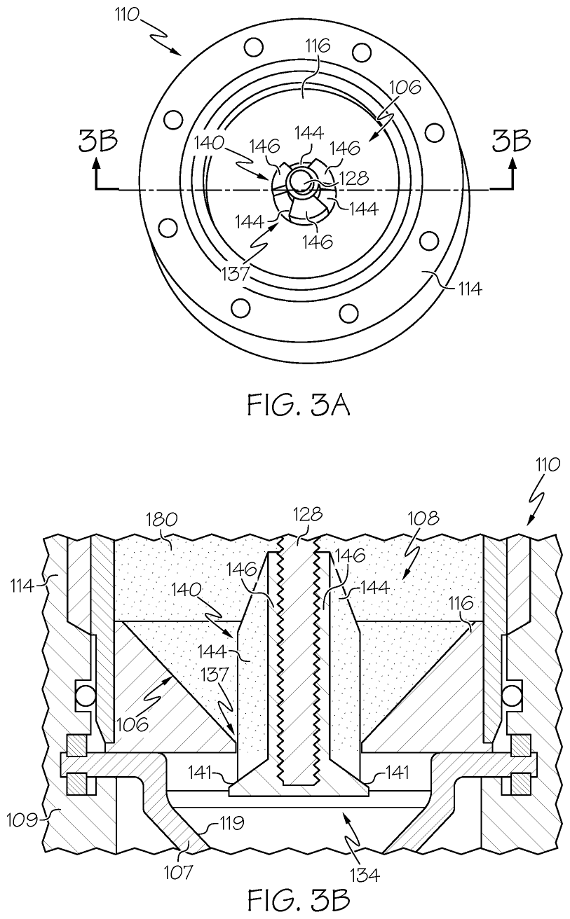

Referring to FIGS. 3A and 3B, valve 140 may be sized, shaped, and/or otherwise configured to meter (e.g., control) a flow rate of agent 180 released from second cavity 108 to third cavity 134. For example, valve 140 may include one or more features and/or surfaces for controlling a flow rate of agent 180 via gravity feeding to enhance continuous steady pressures, volumes, and/or dose rates of agent 180 from valve assembly 100.

Referring specifically to FIG. 3A, valve 140 may include a body having a plurality of ribs 146 positioned adjacent to a plurality of channels 144, with the body of valve 140 being sized, shaped, and/or otherwise configured to be received within center opening 137 of first funnel 106. The plurality of channels 144 and the plurality of ribs 146 may be collectively sized, shaped, and/or otherwise configured to accommodate a metered flow of agent 180 through valve 140. The plurality of channels 144 may extend through valve 140 such that, when valve 140 is moved (repositioned) from second cavity 108 into third cavity 134, agent 180 may flow through center opening 137 via the plurality of channels 144.

The plurality of ribs 146 may be configured to at least partially inhibit the flow of agent 180 though center opening 137, thereby controlling the rate of flow of agent 180 from first funnel 106 to second funnel 107. In the example, valve 140 may include at least three channels 144 and at least three ribs 146. It should be appreciated that valve 140 may include additional and/or fewer channels 144 and/or ribs 146 than those shown and described herein without departing from a scope of this disclosure.

Referring to FIG. 3B, the plurality of channels 144 and the plurality of ribs 146 may be configured to increase the flow rate of agent 180 moving through center opening 137 when valve 140 is moved (repositioned) to the second position. An increase in flow rate may be due to the smaller volumes of agent 180 traveling through each of the plurality of channels 144 as opposed to a single aperture at center opening 137 through which the entire volume of agent 180 may travel.

For example, valve 140 may be configured to deliver agent 180 at flow rates of more than about 0.08 g/s, due to the presence of the plurality of channels 144 and the plurality of ribs 146 within center opening 137. Alternatively, by way of illustrative example, valve 140 may be configured to deliver agent 180 at flow rates of about 0.055 g/s when omitting channels 144 and ribs 146. Agent 180 may have a particle size ranging from about 320 μm to about 740 μm, and valve assembly 100 may be configured to deliver agent 180 at a flow rate of about 5 liters per minute.

In some embodiments, a size and/or diameter of the plurality of channels 144 may at least partially determine the flow rate of the powdered agent (e.g., agent 180) through the system. For example, in embodiments where the plurality of channels 144 may have a relatively larger size, valve assembly 100 may be configured to allow for the powder agent (e.g., agent 180) to achieve a higher flow rate relative to other embodiments where the plurality of channels 144 may have a relatively smaller size. In other embodiments, a size and/or shape of the plurality of ribs 146 may at least partially determine the flow rate of the powdered agent (e.g., agent 180) through the system.

Still referring to FIG. 3B, the plurality of ribs 146 may be configured to enhance a stability of piston rod 128 relative to second cavity 108. For example, the plurality of ribs 146 may be positioned about an exterior perimeter of valve 140, which is disposed about and surrounds a perimeter of piston rod 128. The enhanced stability provided by the plurality of ribs 146 around the exterior perimeter of piston rod 128 may serve to inhibit piston rod 128 from tipping or otherwise moving laterally within second container 110, thereby maintaining piston rod 128 in a concentric position relative to second cavity 108 and/or center opening 137.

In exemplary use, as shown in FIG. 4A, agent 180 may be stored in second container 110, and particularly second cavity 108, prior to an actuation of actuation mechanism 30 (see FIG. 1). Absent the receipt of a pressurized fluid in valve assembly 100, valve assembly 100 may be in the first actuation position with piston assembly 120 in a first position relative to first container 103 and/or second container 110. When in the first actuation position, a respective cavity of first container 103 and/or second container 110 may be at atmospheric pressure levels. With valve 140 coupled to the second (bottom) end of piston rod 128, valve 140 may be positioned in engagement with first funnel 106, and in particular against one or more surfaces defining center opening 137 (e.g., the valve seat). Accordingly, valve 140 may be configured to close and/or seal center opening 137 when valve assembly 100 is in the first actuation position, thereby inhibiting agent 180 from exiting second cavity 108 and entering third cavity 134.

In some embodiments, valve 140 may be sized, shaped, and otherwise configured to fit snugly between first funnel 106 and second funnel 107. In some embodiments, valve 140 may have a cross-sectional profile that is substantially similar to a diameter of center opening 137 such that agent 180 is inhibited from flowing through center opening 137 when valve 140 is received therein. Stated differently, top end 141 may be sized, shaped, and/or otherwise configured to form a fluid tight seal with first funnel 106 when valve 140 is in the first position, thereby inhibiting agent 180 from flowing through center opening 137 until piston assembly 120 is moved to the second position. By preventing inadvertent delivery and/or leakage of agent 180 through center opening 137 and into third cavity 134, valve assembly 100 may be configured to inhibit delivery system 10 from clogging, thereby improving the efficiency of the procedure.

Still referring to FIG. 4A, when piston assembly 120 is in the first position, valve 140 may be positioned flush with the second (lower) end of first funnel 106 (e.g., the valve seat), thereby preventing agent 180 from flowing through first funnel 106 and into second funnel 107 via center opening 137. In response to actuating actuation mechanism 30, a pressurized fluid from the pressurized medium source (not shown) may be received in valve assembly 100. At least a first portion of the pressurized fluid may be received at first container 103 via first fluid inlet 102, and at least a second portion of the pressurized fluid may be received at bottom portion 109 of second container 110 via second fluid inlet 104, such as from tubing 130.

With tubing 130 including orifice 132, valve assembly 100 may be configured to generate a pressure differential between the first (unrestricted) portion of pressurized fluid received in cavity 101 and the second (restricted) portion of pressurized fluid received in third cavity 134. Accordingly, the high pressure zone is formed in first container 103 (e.g., cavity 101 above piston 122), and the low pressure zone is formed in top portion 112 beneath piston 122 (e.g., first cavity 105) and/or in second container 110 (e.g., second cavity 108, third cavity 134, and/or chamber 129), with the high pressure zone having a greater pressure level relative to the low pressure zone. Stated differently, the pressurized fluid received in valve assembly 100 from first fluid inlet 102 may have a first pressure level, and the pressurized fluid received in valve assembly 100 from second fluid inlet 104 may have a second pressure level that is less than the first pressure level.

Upon the pressure level within the high pressure zone (e.g., defined by cavity 101) exceeding a predetermined threshold, the pressurized fluid within the high pressure zone may be greater than an opposing (upward) biasing force generated by biasing mechanism 124 (e.g., a spring) against piston 122. In other words, the predetermined threshold may correspond to a downward force that is greater than the opposing (upward) biasing force of biasing mechanism 124. In some embodiments, the high pressure zone may have a pressure level within a range of about 20 PSI to about 30 PSI, while the low pressure zone (e.g., defined by first cavity 105, second cavity 108, third cavity 134, and/or chamber 129) may have a pressure level within a range of about 14 PSI to about 25 PSI, when valve assembly 100 receives the pressurized fluid via fluid inlets 102, 104. For example, a pressure level within the high pressure zone may be about 21 psi, while a pressure level within the low pressure zone may be about 16 psi. In some embodiments, a biasing force of biasing mechanism 124 may be about 20 lb/inch.

Accordingly, the first portion of pressurized fluid may cause piston assembly 120 to move downward relative to first container 103 and second container 110 from the first position (FIG. 4A) to a second position (FIG. 4B). For example, the first portion of pressurized fluid may urge piston 122 in a downward direction relative to first container 103 (e.g., towards second cavity 108), thereby counteracting the bias generated by biasing mechanism 124, and compressing biasing mechanism 124. Piston rod 128 and valve 140 may move downward relative to second cavity 108 (e.g., towards third cavity 134) as piston 122 is urged downward relative to first cavity 105. In other words, piston rod 128 and valve 140 may be repositioned from the first position to the second position.

As shown in FIG. 4B, when piston assembly 120 is in the second position, valve 140 may be pushed downward into third cavity 134 (towards second funnel 107), thereby opening center opening 137. In the example, top end 141 of valve 140 may disengage one or more surfaces of first funnel 106 (e.g., the valve seat) when piston assembly 120 is moved to the second position, thereby opening center opening 137. Top end 141 may be sized, shaped, and/or otherwise configured to guide agent 180 into third cavity 134 upon valve 140 moving to the second position. For example, top end 141 may include an angled configuration that guides agent 180 toward inner surface 133 of second funnel 107 upon valve 140 moving to the second position, thereby facilitating a continuous flow of agent 180 into third cavity 134.

As agent 180 flows into second funnel 107 (via porous portion 121), the pressurized fluid received within third cavity 134 may mix with agent 180, thereby agitating (e.g., mixing with) agent 180 within second funnel 107. Upon the pressurized fluid agitating agent 180 within third cavity 134, second funnel 107 may be configured to guide a mixture of the agitated agent 180 and pressurized fluid into tubing 118 via center opening 135, and through catheter 36 (FIG. 1) via outlet 190. In other words, the pressurized fluid may mix with agent 180 within third cavity 134, and the mixture of pressurized fluid and agent 180 may simultaneously exit valve assembly 100 via tubing 118. In other embodiments, at least a portion of the pressurized fluid may be guided (upward) through center opening 137 to mix with agent 180 within second cavity 108 when valve 140 disengages the valve seat of first funnel 106.

Following delivery of agent 180 to the target treatment site within the patient, a user may cease actuation of actuation mechanism 30 (see FIG. 1) to thereby cease further delivery of the pressurized fluid to valve assembly 100. In this instance, the pressure level within each of the high pressure zone and the low pressure zone may equalize relative to one another (e.g., return to an atmospheric pressure level). In other words, valve assembly 100 may become depressurized upon terminating delivery of the first and second portions of pressurized fluid to first container 103 and second container 110, respectively.

Accordingly, the pressure level within the high pressure zone may no longer be relatively greater than the pressure level within the low pressure zone, and/or greater than the (upward) biasing force of biasing mechanism 124. As such, the biasing force of biasing mechanism 124 may urge piston 122 upward towards cavity 101 as biasing mechanism 124 transitions from the compressed state to the expanded state. Piston assembly 120 may move from the second position (FIG. 4B) to the first position (FIG. 4A) upon expansion of biasing mechanism 124, thereby causing valve 140 to reengage the valve seat of first funnel 106. With center opening 137 closed by valve 140, piston assembly 120 may be configured to fluidly seal agent 180 within second cavity 108.

In some embodiments, the first portion of pressurized fluid received within first container 103 (from first fluid inlet 102), and the second portion of pressurized fluid received within second container 110 (from second fluid inlet 104), may be vented from valve assembly 100 upon ceasing further actuation of actuation mechanism 30. A size and/or shape of orifice 132 within tubing 130 may control the rate of ventilation of the pressurized fluid within valve assembly 100. For example, at least the first portion of pressurized fluid received within first container 103 may be vented out of valve assembly 100 via first fluid inlet 102, and at least the second portion of pressurized fluid received within second container 110 may be vented out of valve assembly 100 via second fluid inlet 104, such as at orifice 132.

If further delivery of agent 180 to the target treatment site is desired, actuation mechanism 30 may be repeatedly actuated to deliver pressurized fluid to valve assembly 100. Once actuation mechanism 30 is released, valve assembly 100 may become depressurized, thereby preventing delivery of agent 180. It should be appreciated that valve assembly 100 may be configured to provide a pre-flow of pressurized fluid through tubing 118 (and ultimately catheter 36) prior to delivering a mixture of the pressurized fluid and agent 180 to the target treatment site of a patient.

For example, upon an initial actuation of actuation mechanism 30, at least a portion of the pressurized fluid received within bottom portion 109 from second fluid inlet 104 may be guided toward tubing 118 as valve assembly 100 generates the pressure differential between the high pressure zone and the low pressure zone. Accordingly, valve assembly 100 may be configured to deliver the pre-flow of pressurized fluid through tubing 118 and catheter 36, thereby clearing any residual material (e.g., agent 180, fluid, water, or bodily fluids) within second funnel 107, tubing 118, and/or catheter 36 prior to valve assembly 100 moving from the first actuation position (FIG. 4A) to the second actuation position (FIG. 4B).

With the pre-flow of pressurized fluid moving through bottom portion 109 and into tubing 118, valve assembly 100 may be further configured to inhibit delivery stalling of agent 180 once the pressure differential is generated and valve assembly 100 is moved to the second actuation position (FIG. 4B). In this instance, with valve 140 disengaged from the valve seat of first funnel 106 and the pre-flow of pressurized fluid already traveling through bottom portion 109 and into tubing 118, valve assembly 100 may be configured to minimize a delay and/or stalling in delivering agent 180 as agent 180 begins to move into third cavity 134. Instead, agent 180 may interact with the pre-flow of pressurized fluid that is being guided through bottom portion 109 and into tubing 118, thereby enhancing an agitation and delivery of agent 180 to the target treatment site with minimal and/or negligible delay. Accordingly, an instantaneous delivery of agent 180 from delivery system 10, upon actuation of actuation mechanism 30, may be enhanced during a procedure.

It should be further appreciated that valve assembly 100 may be configured to provide a post-flow of pressurized fluid through tubing 118 (and ultimately catheter 36) after delivering a mixture of the pressurized fluid and agent 180 to the target treatment site of a patient. For example, upon ceasing actuation of actuation mechanism 30, at least a portion of the pressurized fluid received within bottom portion 109 from second fluid inlet 104 may be continuously guided toward tubing 118 as the pressure differential between the high pressure zone and the low pressure zone is equalized (e.g., during a depressurization of valve assembly 100). Accordingly, valve assembly 100 may be configured to deliver the post-flow of pressurized fluid through tubing 118 and catheter 36 without agent 180, thereby clearing any residual material (e.g., agent 180) within second funnel 107, tubing 118, and/or catheter 36 as valve assembly 100 is moved from the second actuation position (FIG. 4B) to the first actuation position (FIG. 4A).

Although valve assembly 100 has been shown and described herein as having a pneumatic system (e.g., the pressurized medium source), it should be appreciated that various other suitable systems and/or devices may be utilized to provide a corresponding movement of piston assembly 120, and particularly piston 122, biasing mechanism 124, piston rod 128, and/or valve 140 (e.g., hydraulic systems, mechanical linkages, actuators, electromechanical systems, etc.).

For example, FIGS. 5A and 5B show one or more internal components of delivery system 10 with handle body 12 omitted for illustrative purposes only. In the example, delivery system 10 may include a mechanical actuation assembly 500 that may be coupled to valve assembly 100 shown and described above. Accordingly, like reference numerals are used to identify like components of valve assembly 100. Mechanical actuation assembly 500 may be movably coupled to actuation mechanism 30 and a pressurized medium source 530 of delivery system 10. As described herein, mechanical actuation assembly 500 may be movably coupled to actuation mechanism 30 via one or more links (e.g., bars, rods, cables, etc.) and/or pivot joints (e.g., pins, gears, etc.).

Referring specifically to FIG. 5A, mechanical actuation assembly 500 may further include a movable rod 507 that is coupled to one or more components of valve assembly 100, such as piston assembly 120. Accordingly, mechanical actuation assembly 500 may be configured to move piston assembly 120 from a first position to a second position (as described in detail above) in response to a corresponding movement of movable rod 507 upon actuation of actuation mechanism 30. It should be appreciated that with piston assembly 120 coupled to actuation mechanism 30 via mechanical actuation assembly 500, valve assembly 100 may be configured to transition between the first actuation position (FIG. 4A) and the second actuation position (FIG. 4B) via movement of movable rod 507.

In the example, one or more components of piston assembly 120, such as piston 122 disposed within cavity 101 and/or first cavity 105, may be coupled to movable rod 507 such that movement of movable rod 507 may provide for a corresponding movement of piston 122 relative to cavity 101 and/or first cavity 105. For example, movable rod 507 may extend into first container 103 and/or second container 110. Accordingly, movable rod 507 may be at least partially disposed within cavity 101 and/or first cavity 105 for interacting with piston assembly 120.

As best seen in FIG. 5B, mechanical actuation assembly 500 may include at least a first pivot joint 502 and a plurality of links 504. The plurality of links 504 may be movably coupled to actuation mechanism 30 via first pivot joint 502, such that the plurality of links 504 may be configured to move in response to a movement of actuation mechanism 30 about first pivot joint 502. The plurality of links 504 may be further coupled to movable rod 507, such that the plurality of links 504 may be configured to move movable rod 507 in response to a movement of actuation mechanism 30 about first pivot joint 502.

In the example, mechanical actuation assembly 500 (and particularly the plurality of links 504) may be configured to convert a movement of actuation mechanism 30 in a first direction (e.g., about first pivot joint 502) to a corresponding movement of movable rod 507 in a second direction that may be different than the first direction. In some embodiments, the plurality of links 504 may convert a lateral movement of actuation mechanism 30 relative to handle body 12 (see FIG. 1) into a longitudinal movement of movable rod 507 relative to valve assembly 100. The plurality of links 504 may include a series of rods, bars, cables, pivot joints or pins, gears, rack(s) and pinion(s), and/or various other suitable mechanisms for translating movement of actuation mechanism 30 to a corresponding movement of movable rod 507.

Still referring to FIGS. 5A and 5B, mechanical actuation assembly 500 may include a cam 506 movably coupled to actuation mechanism 30. Cam 506 may be configured to move (e.g., translate, pivot, etc.) in response to an actuation of actuation mechanism 30. In some embodiments, cam 506 may be configured to fluidly couple pressurized medium source 530 to a fluid pressure regulator 540 of delivery system 10 and/or valve assembly 100 (e.g., via tubing 130 and second fluid inlet 104). For example, cam 506 may be configured to interface with and/or engage a portion of pressurized medium source 530 (e.g., a fluid seal) to release the pressurized fluid stored therein for delivery toward pressure regulator 540 and/or valve assembly 100. In some examples, cam 506 may include a rod and/or needle (not shown) coupled thereto, such that movement of cam 506 may provide for a corresponding movement of the rod and/or needle towards pressurized medium source 530 for interacting (e.g., piercing, puncturing, breaking) the fluid seal.

Pressure regulator 540 may be operable to receive and regulate (e.g., decrease) a flow rate and/or a pressure level of the pressurized fluid released from pressure medium source 530 prior to guiding the pressurized fluid to valve assembly 100 (via tubing 130 and second fluid inlet 104). Accordingly, actuation of actuation mechanism 30 may provide for a simultaneous movement of cam 506 (for releasing the pressurized fluid from pressurized medium source 530) and movable rod 507 via the plurality of links 504 (for moving piston assembly 120 to release agent 180). With mechanical actuation assembly 500 configured and operable to transition valve assembly 100 between the first actuation position (FIG. 4A) and the second actuation position (FIG. 4B), only a single portion of pressurized fluid is received within valve assembly 100 for purposes of fluidizing (e.g., agitating, mixing) agent 180.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A valve assembly for a medical device, comprising:
a container including a first region and a second region, wherein the container is configured to house an agent within the second region;
a first fluid inlet fluidly coupled to the first region, the first fluid inlet configured to deliver a first portion of a pressurized fluid to the first region, the first portion of the pressurized fluid having a first pressure level;
a second fluid inlet fluidly coupled to the second region, the second fluid inlet configured to deliver a second portion of the pressurized fluid to the second region, the second portion of the pressurized fluid having a second pressure level that is less than the first pressure level; and
a piston assembly disposed within the container, the piston assembly configured to move relative to the container in response to the first region receiving the first portion of the pressurized fluid and the second region receiving the second portion of the pressurized fluid, wherein the piston assembly includes a valve configured to move from a first position to a second position in response to the piston assembly moving relative to the container;
wherein, in the first position, the valve is configured to inhibit the agent from exiting the second region of the container, and in the second position, the valve is configured to guide the agent out of the second region of the container for delivery from the medical device.

2. The valve assembly of claim 1, further comprising a first funnel positioned within the second region, wherein the first funnel is configured to receive the agent when the valve is in the first position.

3. The valve assembly of claim 2, further comprising a second funnel positioned within the second region relatively below the first funnel, wherein the second funnel is configured to receive the agent from the first funnel when the valve is in the second position.

4. The valve assembly of claim 3, wherein the second funnel is in fluid communication with a source of the pressurized fluid via the second fluid inlet, and the second funnel includes a porous portion along a wall of the second funnel.

5. The valve assembly of claim 4, wherein the second funnel is configured to mix the agent received from the first funnel with the second portion of pressurized fluid received from the second fluid inlet through the porous portion.

6. The valve assembly of claim 5, wherein the second funnel is configured to guide a mixture of the agent and the second portion of pressurized fluid to a delivery conduit of the medical device that is in fluid communication with the second region of the container.

7. The valve assembly of claim 1, wherein the container is configured to generate the first pressure level of the first portion of pressurized fluid within the first region, and the second pressure level of the second portion of pressurized fluid within the second region, thereby forming a pressure differential between the first region and the second region.

8. The valve assembly of claim 7, wherein the piston assembly includes a piston that is at least partially disposed within the first region and a piston rod that is at least partially disposed within the second region, wherein the piston rod has a first end coupled to the valve and a second end coupled to the piston;
wherein the piston assembly includes a biasing mechanism configured to bias the piston towards a first direction, thereby moving the piston rod and the valve to the first position.

9. The valve assembly of claim 8, wherein, in response to forming the pressure differential between the first region and the second region, the valve assembly is configured to counteract the bias generated against the piston by the biasing mechanism to move the piston rod and the valve from the first position to the second position.

10. The valve assembly of claim 7, further comprising a tubing coupled to the second fluid inlet, the tubing is configured to deliver the second portion of pressurized fluid to the second fluid inlet;
wherein the tubing includes at least one orifice that is configured to restrict a flow of the second portion of pressurized fluid through the second fluid inlet.

11. The valve assembly of claim 10, wherein the at least one orifice is configured to regulate the second portion of pressurized fluid to the second pressure level, thereby generating the pressure differential between the first region and the second region.

12. The valve assembly of claim 10, wherein the at least one orifice is configured to vent at least the second portion of pressurized fluid into a surrounding atmosphere of the medical device.

13. The valve assembly of claim 8, wherein the valve includes a plurality of channels configured to regulate a flow rate of the agent moving through the second region when the valve is in the second position.

14. The valve assembly of claim 13, wherein each of the plurality of channels is separated from an adjacent channel by at least one of a plurality of ribs.

15. The valve assembly of claim 14, wherein each of the plurality of ribs is configured to inhibit lateral movement of the piston rod relative to the second region of the container.

16. A valve assembly for delivering an agent, comprising:
an actuator;
a container configured to store an agent;
a fluid inlet fluidly coupled to the container;
a piston assembly disposed within and movable relative to the container, the piston assembly including a valve that is configured to release the agent from the container upon moving from a first position to a second position; and
an actuation assembly coupled to the actuator, the piston assembly, and a source of pressurized fluid;
wherein, in response to actuating the actuator, the actuation assembly is configured to:
interact with the source of pressurized fluid, thereby releasing the pressurized fluid from the source into the container via the fluid inlet; and
move the piston assembly within the container, thereby repositioning the valve from the first position to the second position and releasing the agent from the container;
wherein the pressurized fluid is configured to mix with the agent within the container and guide a mixture of the agent and the pressurized fluid towards an outlet that is in fluid communication with the container.

17. The valve assembly of claim 16, wherein the actuation assembly includes a cam coupled to the source of pressurized fluid and at least one movable rod coupled to the piston assembly;
wherein the cam is configured to engage a seal of the source of pressurized fluid to release the pressurized fluid, and the at least one movable rod is configured to push the valve towards the second position.

18. The valve assembly of claim 16, wherein the valve includes a plurality of channels and a plurality of ribs disposed about a perimeter of the valve, wherein at least one of the plurality of ribs is positioned between an adjacent pair of the plurality of channels.

19. The valve assembly of claim 18, wherein the plurality of channels is configured to control a rate of flow of the agent moving through the valve when in the second position.

20. A method for delivering an agent from a medical device, comprising:
releasing a pressurized fluid into a first region of a valve assembly of the medical device to pressurize the first region to a first pressure level, and a second region of the valve assembly to pressurize the second region to a second pressure level that is less than the first pressure level, wherein the second region houses the agent;
generating a pressure differential between the first region and the second region in response to the first region receiving the pressurized fluid at the first pressure level and the second region receiving the pressurized fluid at the second pressure level;
causing a piston assembly to move relative to the first region and the second region in response to the valve assembly generating the pressure differential between the first region and the second region, wherein the piston assembly includes a valve that is configured to move from a first position to a second position when the pressure differential is generated within the valve assembly; and
releasing the agent from the second region when the valve is moved to the second position, thereby allowing the pressurized fluid and the agent to mix prior to delivery from the medical device.
